(19) United States
(12) Patent Application Publication
(10) Pub. No.: US 2014/0114373 A1
(43) Pub. Date: Apr. 24, 2014

(54) Intermediate Coupler to Facilitate Charging in an Implantable Medical Device System

(71) Applicant: Boston Scientific Neuromodulation Corporation, Valencia, CA (US)

(72) Inventor: Daniel Aghassian, Glendale, CA (US)

(21) Appl. No.: 13/941,978

(22) Filed: Jul. 15, 2013

Related U.S. Application Data
(60) Provisional application No. 61/716,662, filed on Oct. 22, 2012.

(51) Int. Cl. A61N 1/378

(52) U.S. Cl. CPC A61N 1/3787
USPC 607/45

(57) ABSTRACT

An intermediate coupler is used to improve inductive coupling between an external charger and an implantable medical device having a battery requiring charging. The intermediate coupler comprises a coil (inductor) coupled to a capacitor, whose values are chosen to resonate at the frequency of the magnetic field emitted by the external charger. The intermediate coupler preferably contains no power source such as a battery, and can operate passively. When the intermediate coupler receives the magnetic field from the external charger, a current is induced in its coil, and the intermediate coupler generates its own magnetic field which is captured by the implantable medical device and used to charge its battery.

### CROSS REFERENCE TO RELATED APPLICATIONS

This is a non-provisional of U.S. Provisional Patent Application Ser. No. 61/716,662, filed Oct. 22, 2012, which is incorporated by reference and to which priority is claimed.

### FIELD OF THE INVENTION

The present invention relates to wireless external charging of an implantable medical device.

### BACKGROUND

Implantable stimulation devices are devices that generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system.

As shown in FIGS. 1A-1C, a SCS system typically includes an Implantable Pulse Generator (IPG) 100, which includes a biocompatible device case 30 formed of a conductive material such as titanium for example. The case 30 typically holds the circuitry and battery 26 necessary for the IPG to function, although IPGs can also be powered via external RF energy and without a battery. The IPG 100 is coupled to electrodes 106 via one or more electrode leads (two such leads 102 and 104 are shown), such that the electrodes 106 form an electrode array 110. The electrodes 106 are carried on a flexible body 108, which also houses the individual signal wires 112 and 114 coupled to each electrode. In the illustrated embodiment, there are eight electrodes on lead 102, labeled E₁-E₈, and eight electrodes on lead 104, labeled E₉-E₁₆, although the number of leads and electrodes is application specific and therefore can vary. The leads 102, 104 couple to the IPG 100 using lead connectors 38a and 38b, which are fixed in a non-conductive header material 36, which can comprise an epoxy for example.

As shown in the cross-section of FIG. 1C, the IPG 100 typically includes an electronic substrate assembly including a printed circuit board (PCB) 16 containing various electronic components 20. Two coils (more generally, antennas) are generally present in the IPG 100: a telemetry coil 13 for transmitting/receiving data to/from an external controller (not shown); and a charging coil 18 for charging or recharging the IPG's battery 26 using an external charger 50 (discussed further below). In this example, the telemetry coil 13 and charging coil 18 are within the case 30, as disclosed in U.S. Patent Publication 2011/0112610. (FIG. 1B shows the IPG 100 with the case 30 removed to ease the viewing of the two coils 13 and 18). However, the telemetry coil 13 may also be mounted within the header 36 of the IPG 100 (not shown).

FIG. 2A shows a plan view of the external charger 50, and FIG. 2B shows the external charger 50 wirelessly providing power (magnetic field B1) to recharge the IPG 100's battery 26. The external charger 50, like the IPG 100, also contains a PCB 70 on which electronic components 72 are placed, some of which are discussed subsequently. A user interface 74, including touchable buttons and perhaps a display, LEDs, and/or a speaker, allows a patient or clinician to operate the external charger 50. A battery 76 provides power for the external charger 50, which battery 76 may itself be rechargeable. The external charger 50 can also receive AC power from a wall plug. An appropriately sized hand-holdable or body-wearable case 77 contains all of the components.

Power transmission from the external charger 50 to the IPG 100 occurs wirelessly, and transcutaneously through a patient's tissue 25, via inductive coupling. FIG. 3 shows details of the circuitry used to implement such functionality. A coil 17 in the external charger 50 is energized via charging circuit 122 with a constant non-data-modulated AC current, Icharge, to create an AC magnetic charging field. This magnetic field induces a current in the charging coil 18 within the IPG 100, which current is rectified (132) to DC levels, and used to recharge the battery 26, perhaps via a charging and battery protection circuit 134 as shown. The frequency of the magnetic charging field can be perhaps 80 kHz or so, but this frequency could be different or could comprise a range of frequencies capable of transcutaneous transmission. When charging the battery 26 in this manner, is it typical that the case 77 of the external charger 50 touches the patient's tissue 25, although this is not strictly necessary.

The IPG 100 can also communicate data back to the external charger 50 during charging using reflected impedance modulation, which is sometimes known in the art as Load Shift Keying (LSK). Such back telemetry from the IPG 100 can provide useful data concerning charging to the external charger 50, such as the capacity of the battery 26, or whether charging is complete and the external charger 50 can cease. LSK telemetry is described further in U.S. patent application Ser. No. 13/608,666, filed Sep. 10, 2012, which the reader is assumed familiar.

An issue arising when inductive coupling is used for power transmission to the IPG, and IPG battery 26 charging more specifically, relates to the coupling between the coils 17 and 18 in external charger 50 and the IPG 100. Coupling, generally speaking, comprises the extent to which energy expended at the transmitting coil 17 in the external charger 50 is received at the charging coil 18 in the IPG 100. It is generally desired that the coupling between coils 17 and 18 be as high as possible: higher coupling results in faster charging of the IPG battery 26 with the least expenditure of energy in the external charger 50. Poor coupling is disfavored, as this will require high energy drain (i.e., a high Icharge) in the external charger 50 to adequately charge the IPG battery 26, and/or slow (or non-existent) charging of the battery 26. As explained in the above-referenced '666 application, coupling depends on many variables, such as the permeability of the environment, and the relative geometry of the coils 17 and 18. If the coils are misaligned, that is if the coils are: at a great distance, such as distance “d” shown in FIG. 4A; not co-axial, such as offset distance “x” shown in FIG. 4B; or not co-planar, such as offset angle “θ” shown in FIG. 4C, coupling will be reduced.

It is known to place the external charger 50 of FIGS. 2A and 2B in a belt (sometimes known as a “fanny pack”), which will hold the external charger 50 in close proximity to a patient's SCS IPG 100 during a charging session. Even if there is some degree of misalignment between the external charger 50 and the IPG 100, the alignment will generally be sufficient to charge the implant, even if not ideally quick. But this is not true in all cases: the belt may not be properly positioned by the patient relative to the IPG 100, or such relative positioning might change during the charging session, such as when the patient moves.

If coupling is poor, or is eventually rendered poor during a charging session, the patient is typically required to move the external charger 50 (or the belt in which it is held) in the hopes that the external charger 50 can be put into better alignment with the IPG 100. Thus, the external charger 50 may issue an audible tone, or provide some other indication to the patient, when poor coupling is detected. See, e.g., U.S. patent application Ser. No. 13/608,600, filed Sep. 10, 2012. Reliance on the patient to adjust the positioning of the external charger 50 relative to the IPG 100 can be problematic, as the patient is inconvenienced and may not be able to make the required adjustment of the position of the external charger 50.

A particular problem with charging by inductive coupling relates to the distance at which such method is effective. As discussed in U.S. Patent Application Publication 2012/0004709, the strength of an inductive magnetic field received at coil 18 typically reduces proportional to the cube of the distance (d) from the generating coil 17. As a result, the distance at which an external charger such as 50 can effectively charge an IPG 100 is typically less than one meter, and can be even smaller depending on the degree of coil-to-coil misalignment.

The inventor desires to increase the effective charging distance, and more generally increase the effective coupling, between an external charger and an IPG. The inventor also desired to provide a passive charging solution in which a patient's implant can be charged with minimal or no involvement on the patient's part. Means for achieving these and other beneficial goals are disclosed.

### BRIEF DESCRIPTION OF THE DRAWINGS

FIGS. 1A-1C show different views of an implantable medical device, specifically an Implantable Pulse Generator (IPG).

FIGS. 2A and 2B show a prior art external charger used to charge a battery of the IPG.

FIG. 3 shows the circuitry of the external charger and the IPG relevant to charging the IPG battery.

FIGS. 4A-4C show various ways in which the coils in the external charger and the IPG can be misaligned.

FIG. 5 shows an intermediate coupler in accordance with the invention that can be used with an external charger to facilitate charging of an IPG battery.

FIGS. 6A and 6B show how the intermediate coupler can be used to extend the range at which the IPG can be charged by the external charger.

FIGS. 7A and 7B show how the intermediate coupler can function to facilitate charging of an IPG battery even if not perfectly aligned, or behind the IPG from the vantage of the external charger.

FIG. 8 shows an intermediate coupler incorporated into a bed, and use of a floor-based external charger.

FIG. 9 shows a cross section of the construction of the intermediate coupler.

FIGS. 10A-10C show use of the intermediate coupler with a hand-holdable or body-wearable external charger, and incorporation of the intermediate coupler into a charging belt.

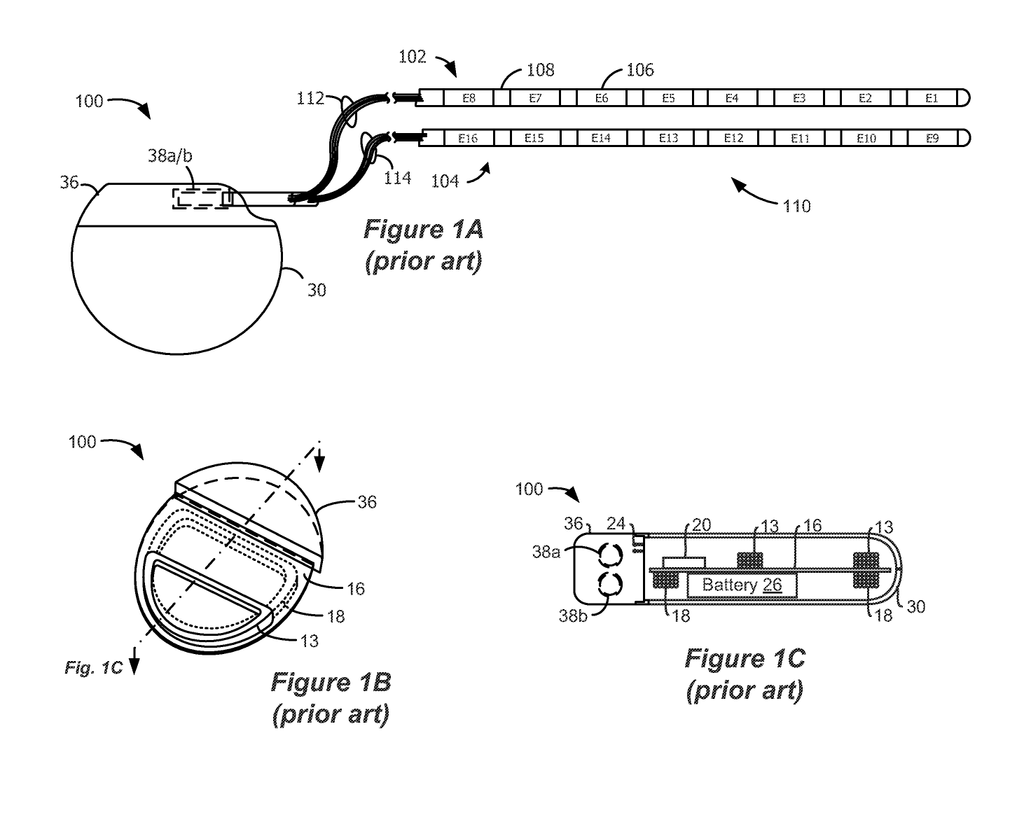

### DETAILED DESCRIPTION

An intermediate coupler is used to improve inductive coupling between an external charger and an implantable medical device having a battery requiring charging. The intermediate coupler comprises a coil (inductor) coupled to a capacitor, whose values are chosen to resonate at the frequency of the magnetic field emitted by the external charger. The intermediate coupler preferably contains no power source such as a battery, and can operate passively. When the intermediate coupler receives the magnetic field from the external charger, a current is induced in its coil, and the intermediate coupler generates its own magnetic field which is captured by the implantable medical device and used to charge its battery. Use of the intermediate coupler can extend the range at which the external charger may operate to charge the implant, can improve the effective coupling between the external charger and the implant, and can reduce concerns over misalignment between the external charger and the implant. Additionally, the intermediate coupler can be used with passive changing systems that charge a patient's implant with little or no interaction from the patient. Thus, the disclosed charging systems employing an intermediate coupler can make charging simpler and more reliable for patients. This is particularly beneficial where the patient may have difficulty with charging the battery in their implant, such as if the patient is elderly, or suffering from dementia, which patients may have an implanted Deep Brain Stimulator (DBS) requiring charging.

As noted in the Background, charging of an implant by inductive coupling generally occurs at a relatively short distance. Such short distance works fine when, as is typical, the external charger is hand-holdable or body-wearable, and portable, and can be positioned proximate to a patient's implant using a belt for example. Even if there is some degree of misalignment between the external charger and the implant, charging will generally be sufficient.

But the art would benefit from other types of external charging systems as well. For example, the art has realized benefit in having a stationary external charger. U.S. Patent Application Publication 2012/0004709 discloses a stationary external charger that can be placed in a room, on a wall, under a bed, etc.—that is, in locations where an implant patient is expected to spend a significant amount of time. This is beneficial, because a stationary external charger can charge a patient's implant without his or her involvement, and in situations in which operation of a traditional hand-held external charger is impractical, such as when the patient is sleeping.

Distance of the patient from the external charger though is still a concern, particularly when charging by magnetic inductive coupling is involved. The '709 Publication addresses this concern by charging using longer-distance electric fields instead of shorter-distance magnetic fields when charging at longer distances. However, electric fields generally will not provide as much power to the implant, and therefore will not charge the implant as quickly. It is therefore desired to improve the distance, or more generally the coupling, between an external charger and an implant in a magnetic inductive charging system.

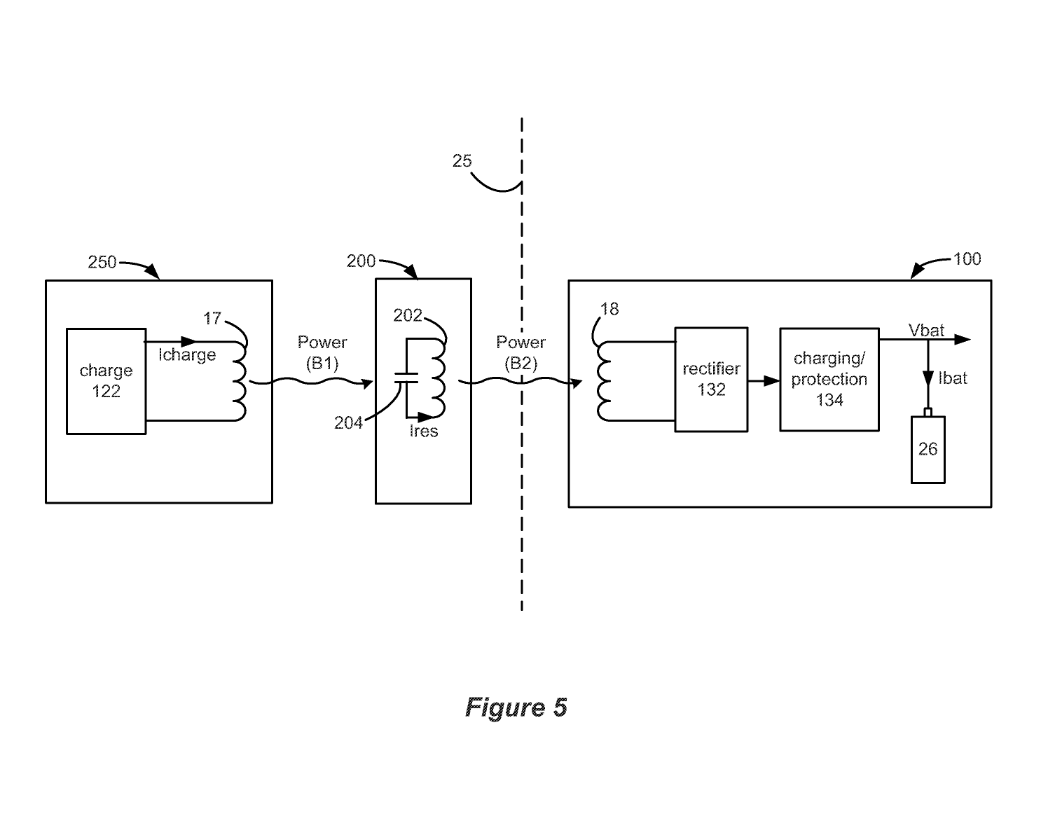

FIG. 5 shows a solution to this problem in the form of an intermediate coupler 200, which as its name implies is used as an intermediary to improve the coupling between an external charger 250 and an IPG 100, and hence increase the distance at which charging can occur.

The intermediate coupler 200 as shown comprises a coil 202 connected in parallel with a capacitor 204, thus forming a resonant circuit. The coil 202 in the intermediate coupler 200 receives power (magnetic field B1) from the external charger 250, which may be the same as the hand-held external charger 50 described previously, or which may be stationary as discussed subsequently. Magnetic field B1 induces an AC current, Ires, in the coil 202. When the values of the inductance of the coil 202 and the capacitance 204 are chosen appropriately, the intermediate coupler 202 will generally resonate at the frequency or range of frequencies of magnetic field B1, e.g., 80 kHz. Such resonance of the intermediate coupler 202 produces a second magnetic field B2. As shown in FIG. 5, this second magnetic field B2 is received by the charging coil 18 in the IPG 100, which can in turn charge the IPG's battery 26 as previously described. Thus, the intermediate coupler 200 receives the charging field produced from the external charger 250, and in turn produces its own charging field to charge the implant. Note that this occurs passively: the intermediate coupler 200 is not powered from a power source, such as a battery or connection to a wall plug. As will be discussed further below, the coil 202 of the intermediate coupler 200 has a relatively large area that is larger than the area of the coil 18 in the IPG 100, and which may also be larger than the coil 17 in the external controller.

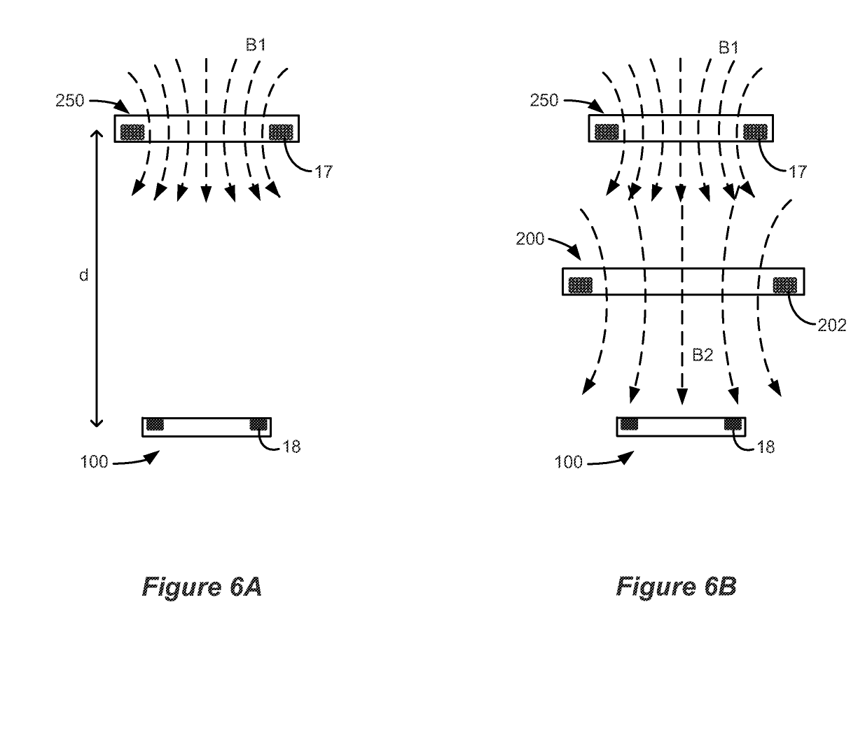

Use of a large-area intermediate coupler 200 to assist in charging the IPG 100 is especially useful given the IPG's relatively small size. The IPG 100, because it is implanted in a patient, must be conveniently small. As a result, the charging coil 18 (typical in the IPG's case 30) will have a small area of perhaps 10 cm² or less. The IPG coil 18 will not capture significant amounts of flux from the magnetic field produced by the external charger 50, particularly at large distances, d. FIG. 6A illustrates this by showing the flux lines of the magnetic field B1 produced by coil 17 in the external charger 250 upon the application of a current (Icharge). As shown, the small coil 18 in the IPG 100 will not capture much of this flux at distance d. However, as shown in FIG. 6B, when the intermediate coupler 200 is positioned between the external charger 250 and the IPG 100, the large area of coil 202 is able to capture more of the external charger's flux, and in turn generate its own magnetic field, B2. If a significant amount of the energy from magnetic field B2 reaches the coil 18 in the IPG 100, it can be suitably charged, where it could not before (FIG. 6A) given its distance to external charger 250. The intermediate coupler 200 thus operates to extend the range, or more generally the effective coupling, between the external charger 250 and the IPG 100.

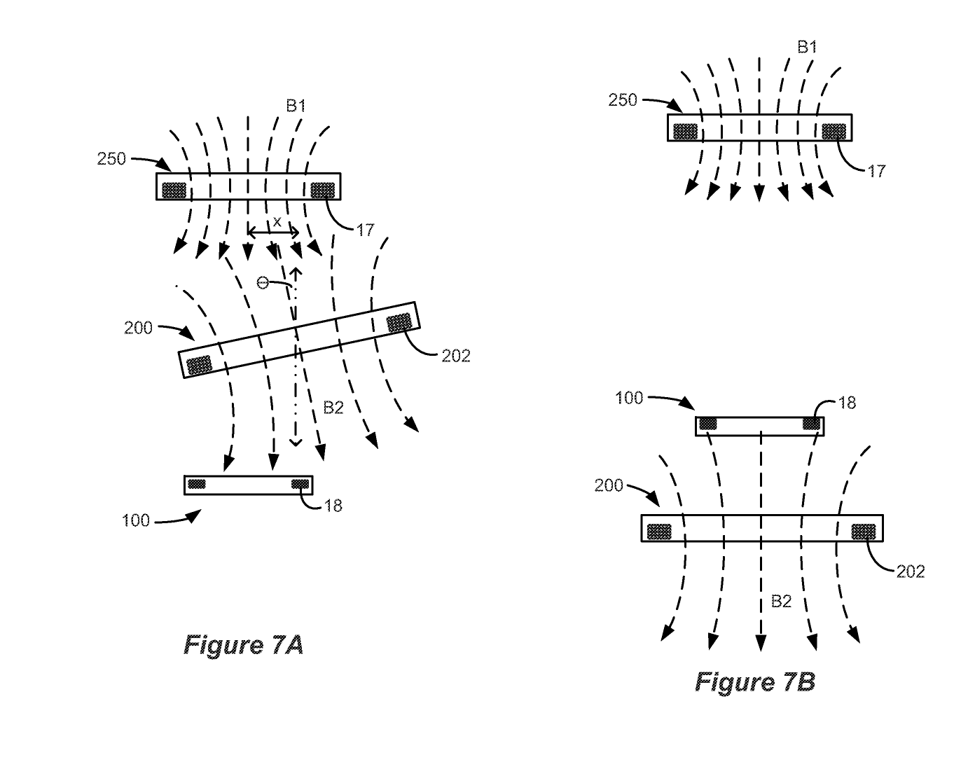

Although the intermediate coupler 200 is shown in FIG. 6B in a generally ideal alignment in which its coil 202 is planar and co-axial with both the coils 17 and 18 in the external charger 250 and the IPG 100, such ideal alignment is not required. For example, in FIG. 7A, the intermediate coupler 200 is both axially offset (x) and not planar (θ) with coils 17 and 18. Nevertheless, the intermediate coupler 202 is still capable of receiving flux from magnetic field B1 produced by the external charger 250, and in turn producing its own magnetic field B2 that reaches the IPG 100, although the effective coupling between the external charger 250 and the IPG 100 would be smaller than for the ideal case of FIG. 6B.

It is also not required that the intermediate coupler 200 be between the external charger 250 and the IPG 100, as shown in FIG. 7B. As shown in the example, the intermediate coupler 200 is behind the IPG 100 from the vantage of the external charger 250. Given the relatively large area of its coil 202, the intermediate coupler 200 may be able to capture significant flux from magnetic field B1, and produce magnetic field B2. Therefore, even though the IPG 100 cannot significantly receive magnetic field B1, it can still receive significant flux from the magnetic field B2 produced by the intermediate coupler 200, particularly if the IPG 100 and intermediate coupler 200 are sufficiently close.

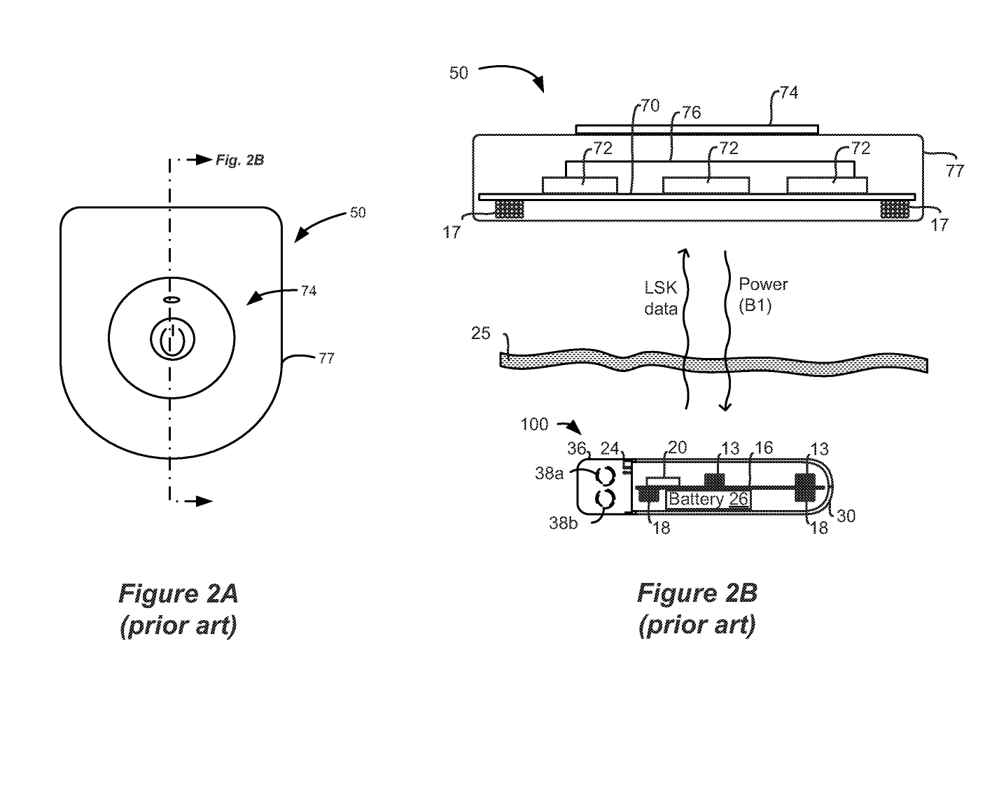

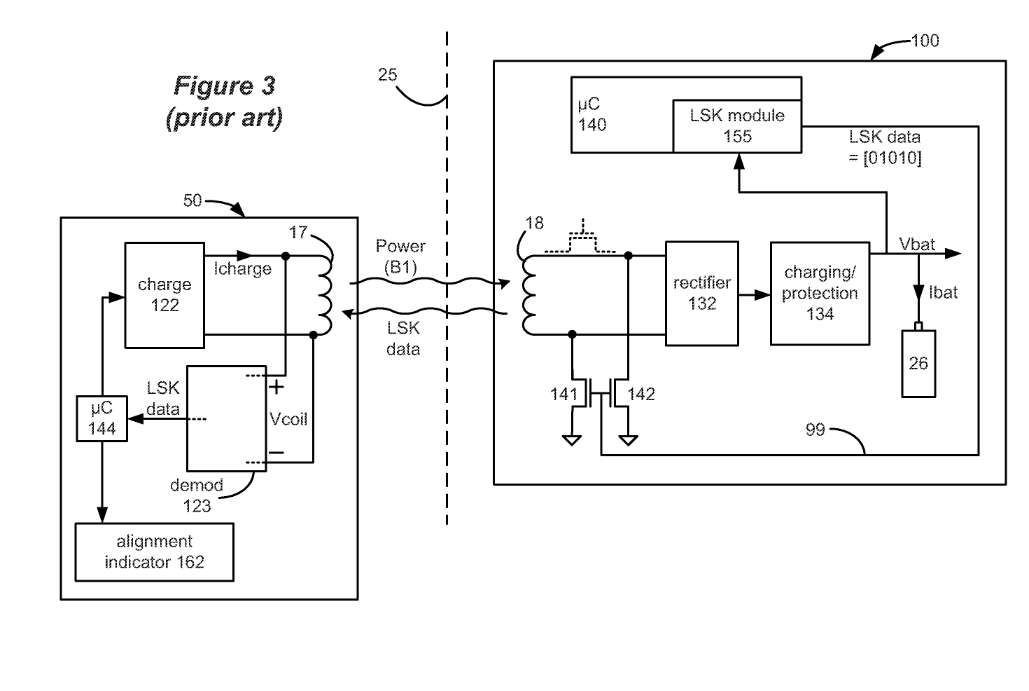

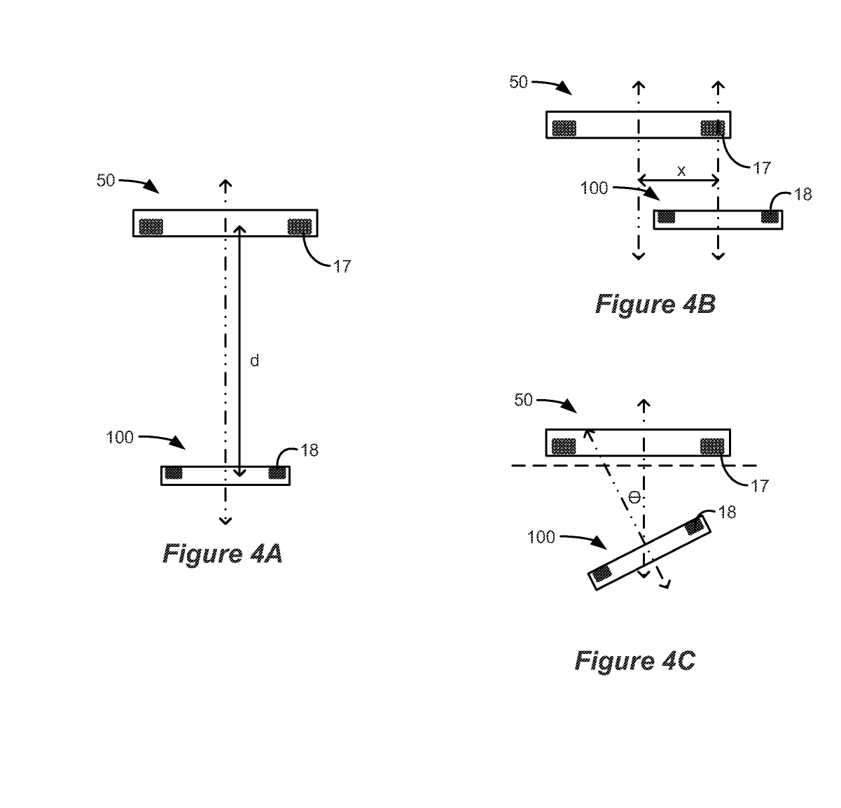

Although the external charger 250 usable with the intermediate coupler 200 can be hand held as shown in FIGS. 2A and 2B, a different design for the external charger 250 can be useful, particularly when the intermediate coupler 200 is used to extend the range of a stationary external charger. FIG. 8 shows an example of a stationary external charger 240 usable with an intermediate coupler 200 incorporated into a bed 260. As shown, the stationary external charger 240 can lie on a floor underneath the bed 260. The stationary external charger 240 would generally be built mechanically and electrically similar to the external charger 50 discussed earlier with respect to FIGS. 2A-3, but would have a larger area. The housing of the stationary external charger 240 may have an on/off button 252 and other user interface aspects such as those discussed earlier. It may plug into a wall outlet at a plug 254, or may be powered by batteries. The coil 17 and circuitry in the stationary external charger 240 can be made thin, and the housing of the external charger 250 may range from 0.3 to 1.0 inches.

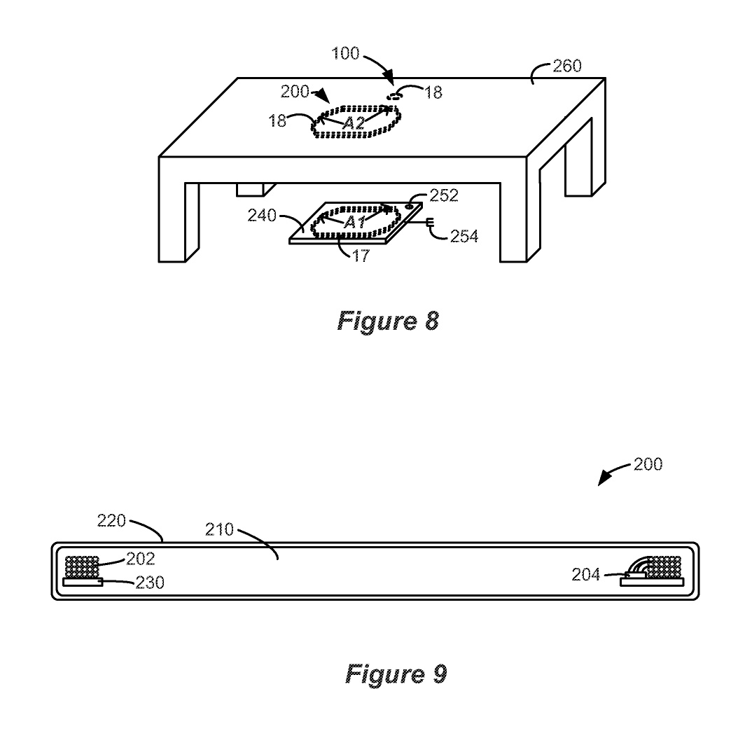

The intermediate coupler 200 can be incorporated into a mattress or pad of the bed 260, or could be formed in its own portable housing, as shown in cross section in FIG. 9. Thus, the intermediate coupler 200 can be integrated or incorporated into a home furnishing in a discreet fashion not noticeable by the user. The ends of coil 202 and soldered to capacitor 204, which would be quite small in comparison. The coil 202 (and possibly also the capacitor 204 after it is soldered) can be coated in epoxy to give the coil 202 some rigidity, or can be coated in rubber to render it somewhat flexible. Alternatively, the coil and capacitor 204 can be mounted to an optional printed circuit board 230. Again, because the intermediate coupler 200 is passive, it need not contain any other components. However, other components could be added, such as some sort of user interface to alert the user whether the intermediate coupler 200 is receiving the stationary external charger 240's magnetic field B1. For example, a small LED (not shown) placed in parallel with the coil 202 and the capacitor 204 could alert the user that the intermediate coupler 200 is receiving energy, with the brightness of the LED indicating qualitatively how much current (Ires; FIG. 5) has been induced in coil 202.

For comfort, the assembly can be encased in padding 210, which may thereafter be encased in a removable, washable cover 220. An intermediate coupler 200 formed in this manner would have a form factor somewhat similar to a heating pad, although lacking a wall plug. The intermediate coupler 200 preferably contains no conductive components other than the coil 202, which reduces magnetic-field induced heating. The intermediate coupler 200 is also light, and thus can be worn by the patient, as described further below. It is also easy and inexpensive to manufacture.

Design of the external charger 250 and intermediate coupler 200 can be made in many different ways. In one example, the external charger 250 comprised a coil 17 having an area A1 of roughly 900 cm² (i.e., a rectangular coil approximately 30 cm on each side). One skilled in the art will understand that the number of windings in coil 17, the inductance and resistance of the coil 17, the AC current passed through the coil 17, can be tailored for the application at hand. The coil 202 in the intermediate coupler 200 can have a somewhat similar area A2 of roughly 700 cm², again with a number of windings, inductance, and resistance tailored for the application at hand. The value of the capacitor 204 in the intermediate coupler 200 is normally chosen in combination with the inductance of coil 202 such that magnetic field B2 generally resonates at the same frequency or frequency range of magnetic field B1.

Choosing particular component values and materials is a matter of design preference, and may be dictated by the manner in which the intermediate coupler 200 is intended to be used. For efficient power transmission, it is desired that both the coil 17 in the external charger 250 and the coil 202 in the intermediate coupler 200 have a high Q value. As one skilled in the art will recognize, this means that frequency spectra of these coils have sharp peaks (e.g., at 80 kHz), which can be achieved by the use of low resistance coils. However, while using high-Q coils promotes efficient power transfer through the intermediate coupler 200, it also makes tuning more of a concern, i.e., to match the peaks of the resonant frequencies of the coils 17 and 202. The stationary external charger 240 can implement tuning circuitry to adjust the frequency of magnetic field B1 to match to the resonance of the intermediate coupler 200. See, e.g., U.S. Patent Publication 2005/0119716. Alternatively, lower-Q coils 17 and 202 with broader resonance peaks can be used, and which can be formed by increasing the series resistance of the coils. While not as efficient, broader spectra reduce tuning concerns.

Informal testing of a prototype system suggested that use of the intermediate coupler 200 can at least extend the distance at which an IPG 100 can be effectively charged by two to four times, roughly to a distance of at least two meters. In one example of such utility of the intermediate coupler 200, a prototype IPG coil 18 was tested at a distance of about 1.5 meters from the external charger 250 prototype discussed above, but did not receive suitable power (see FIG. 6A). However, when the intermediate coupler 200 prototype was held between the two, suitable power was received by the IPG (see FIG. 6B). Performance of the intermediate coupler 200 is improved the closer it is to the IPG 100, because the charging coil 18 in the IPG 100 will receive more flux from the coil 202 in the intermediate coupler 200 in that circumstance. Informally positioning the intermediate coupler 200 in various misaligned fashions (see FIG. 7) also resulted in adequate power reception at the prototype coil 18, as did positioning of the intermediate coupler 200 behind prototype coil 18 (see FIG. 7B).

Power transmission efficiency from the stationary external charger 240 to the IPG 100 through the intermediate coupler 200 may be low in some applications, which may result in slow charging of the IPG's battery. This can be acceptable though in a typical situation in which a stationary external charger 240 is used. As noted above, a stationary external charger 240 is particularly useful to charge an implant in a place where a patient normally spends significant time, and would benefit from charging of his implant, even if charging occurs slower than it would using a portable hand holdable or body wearable external charger 50 (FIGS. 2A & 2B). Power transmission efficiency can be improved by focusing the magnetic field B1 in one direction towards the intermediate coupler 200. This can be accomplished by providing a shield of a high permeability material behind the coil 17 in the stationary external charger 240, which is not shown in the figures but is further discussed in U.S. Patent Publication 2001/0234155, which is incorporated herein by reference in its entirety.

To provide even further benefit and ease of use to the patient, the stationary external charger 240 can detect the presence of the patient or the implant in any known manners, and can automatically turn on to generate a magnetic field, thus relieving the patient of having to press the on/off switch 252 on the stationary external charger 240. For example, the stationary external charger 240 can periodically come out of standby mode to try and detect an IPG 100 in the vicinity, and if one is detected, can start generation of magnetic field B1 to charge the implant though the intermediate coupler 200. This charging field can periodically cease to allow the stationary external charger 240 to query whether the IPG 100 is still present and if it requires further charging. However, ceasing the charging field is not required if the frequencies used for communication are outside of the band of frequencies used for charging.

Although not shown in the figures, the stationary external charger 240 can comprise two orthogonal coils driven 90-degrees out of phase to provide a rotating magnetic field B1, which may provide better coupling to the intermediate coupler 200 so long as the intermediate coupler is reasonably well coupled to either coil. The use of orthogonal coils in an external charger is discussed in U.S. Patent Publication 2012/0004709, with which the reader is assumed familiar.

Use of a floor-based stationary external charger 240 is merely one example. Given the thinness of the stationary external charger 240, it could be positioned in other places or in other home furnishings that would be convenient for charging a patient's implant if in the vicinity. For example, the stationary external charger 240 could be wall mounted, and perhaps even disguised by hiding it behind a wall-mounted picture. It could also be incorporated into a chair, or a chair pad. In other words, the external charger 240 can be incorporated into a home furnishing in a discreet fashion not generally noticeable by the user. Also, two or more stationary external chargers 240 could be used in a particular location to even further broaden the locations in which a patient can receive a suitable charge via the intermediate coupler 200. For example, two stationary external chargers 240 could be placed on orthogonal walls in the room, thus providing good coupling to the intermediate coupler 200 if the axis around which coil 202 is wound is generally pointing to either of the chargers 240.

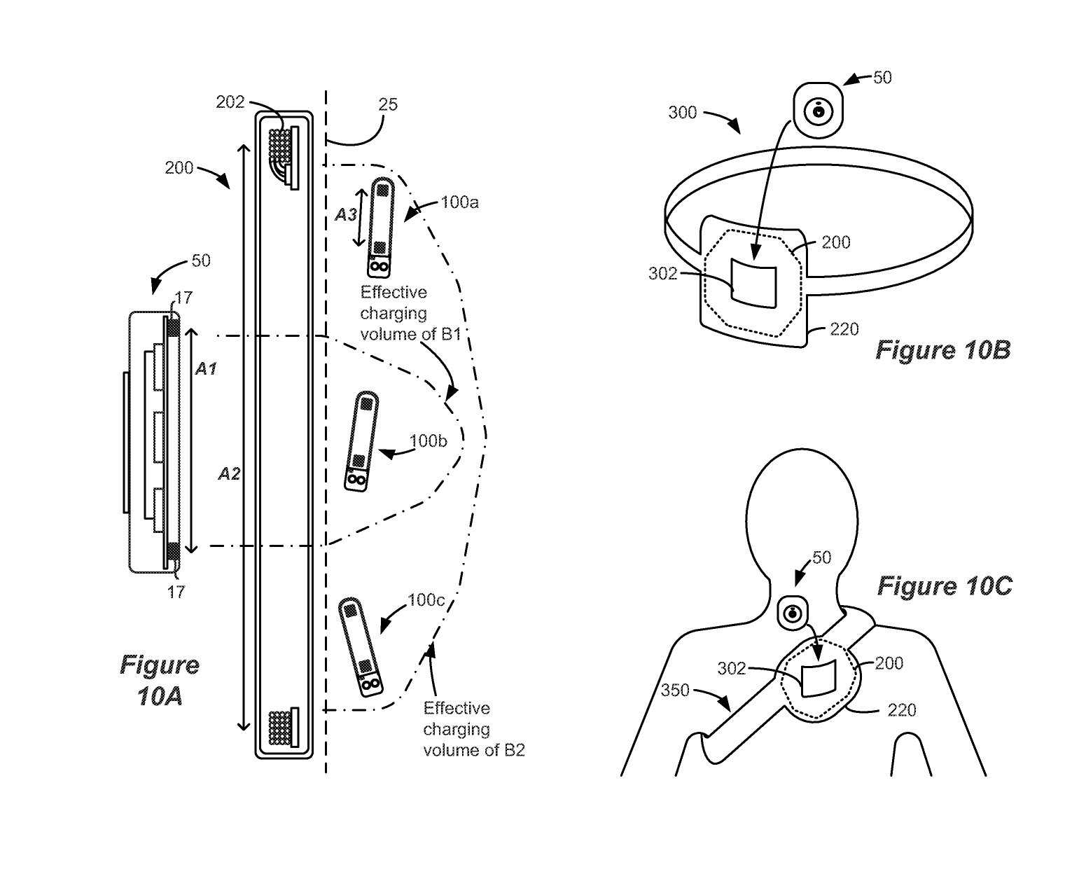

FIG. 10A illustrates another example of the utility of the intermediate coupler 200 to improving implant battery charging, and in particular to alleviate problems of misalignment between an external charger and the IPG. In this example, a traditional hand held external charger 50, such as was illustrated earlier in FIGS. 2A-2B, is used in conjunction with an intermediate coupler 200 to charge an IPG. Three possible positions 100a-100c of the IPG relative to the external charger 50 are shown. The area of coil 17 in the external charger 50 (A1) is typically around 50 cm², and (ignoring the effect of the intermediate coupler 200 for the moment) the effective volume at which this coil 17 can charge an implant (i.e., the effective charging volume of B1) is approximated in dotted lines, and is limited in part by A1. The effective charging volume can be defined with respect to some charging parameter, such as the positions for which the charging rate of the IPG's battery 26 exceeds some minimum threshold.

It is seen that IPG position 100b is within the effective charging volume of B1, largely because that IPG 100b is reasonably coaxial and planar with coil 17, and is not implanted too deep in the patient's tissue 25. IPG positions 100a and 100c, by contrast, are poorly aligned to the coil 17, and are outside of the effective charging volume of B1. In short, IPGs in positions 100a and 100c relative to the external charger 50 will likely not be charged, or will be inadequately charged.

The intermediate coupler 200 addresses this problem and allows IPGs in a wider range of positions relative to the external charger 50 to be adequately charged. The area of the coil 202 in the intermediate coupler 200 (A2) is larger than the area A1 of the coil 17 in the external charger 50, and hence captures a significant portion of the flux of magnetic field B1. As noted earlier, this causes coil 202 in the intermediate coupler 200 to produce its own magnetic field B2, the effective charging volume of which is also shown in dotted lines.

Notice that the larger area A2 of coil 202 produces a larger area for the effective charging volume of B2. The relative depth of the effective charging volume of B2 may not be as great as the depth of the effective charging volume of B1; this is due to losses inherent in the conversion of magnetic field B1 to B2, as well as the fact that coil 202 in the intermediate coupler 200 has a larger area over which the power of B1 is spread. The wider area for the effective charging volume of B2 means that implants in a wider range of positions can be adequately charged compared to the use of the external charger 50 alone. Thus, in the example shown, all of IPG positions 100a-100c will receive an adequate charging field by virtue of B2. Because the power of B1 is spread over the larger area of coil 202, an IPG in position 100b may see less power when the intermediate coupler 200 is used compared to use of the external charger 50 alone. But IPGs in positions 100a and 100c—through the use of intermediate coupler 200—will be suitably charged where they could not have been adequately charged by the use external charger 50 alone.

Thus, by spreading the power created by the external charger 50 over a larger area through the use of the intermediate coupler 200, a wider range of IPGs positions can be adequately charged by the external charger 50. This is of great convenience to the patient and eases use of the external charger 50, as the patient need worry less whether the smaller external charger 50 is adequately aligned with his implant. If needed, the power of the magnetic field B1 produced by the external charger 50 can be increased (i.e., Icharge can be increased) to provide a sufficient magnetic field to charge the IPG 10 anywhere in volume B2.

A particular implementation appreciated by the inventor from the foregoing discussion of FIG. 10A is presented in FIG. 10B. Shown is a belt 300 wearable around a patient's waist of the type typically used to hold the external charger 50 relative to an SCS IPG 100 during a charging session. An intermediate coupler 200 is incorporated into the belt 300, which may be accomplished by attaching or integrating a belt with the cover 220 of the intermediate coupler 200. A pocket 302 is attached to the cover 220 of the intermediate coupler 200 into which the external charge 50 can be inserted. When positioned in the pocket 302, the external charger 50 is positioned with respect to the intermediate coupler 200 as discussed above with respect to FIG. 10A, with the same beneficial results.

FIG. 10B is one example of the use of the intermediate coupler 200 with an external charger 50, and other modifications are possible. For example, although not shown, a belt 300 could have a suitably large pocket 302 into which both the intermediate coupler 200 and the external charger 50 can be inserted, in which case the intermediate coupler 200 would not need to be integrated into the belt 300. Notice that the pocket 302 holds the external charger 50 within an area bounded by the area A2 of the coil 202 of the intermediate coupler.

Similar articles wearable by the patient and positionable over the patient's implantable medical device are possible. For example, a shoulder harness 350 can incorporate an intermediate coupler 200 as shown in FIG. 10C, which is useful to facilitate charging the battery in a chest-positioned implant, such as a Deep Brain Stimulator (DBS), which is typically implanted under a patient's collarbone.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for charging a battery in an implantable medical device of a patient, comprising:
an external charger comprising a first coil, wherein the first coil is configured to emit a first alternating current (AC) magnetic field; and
a coupler comprising a second coil, wherein the second coil is configured to receive the first AC magnetic field and generate a second AC magnetic field in response; and
an implantable medical device comprising a third coil and a battery,
wherein the third coil in the implantable medical device is configured to receive the second AC magnetic field to charge the battery.

2. The system of claim 1, wherein the second coil has a second area, the third coil has a third area, and the second area is larger than the third area.

3. The system of claim 1, wherein the first AC magnetic field and the second AC magnetic field have the same frequency.

4. The system of claim 1, wherein the external charger is hand-holdable or body-wearable, and portable.

5. The system of claim 1, wherein the external charger is stationary.

6. The system of claim 1, wherein the coupler is portable.

7. The system of claim 1, wherein the coupler is integrated into a home furnishing.

8. The system of claim 1, wherein the external charger is integrated into a home furnishing.

9. The system of claim 1, wherein the coupler is incorporated into an article wearable by the patient.

10. The system of claim 1, wherein the coupler does not contain a battery or a wall plug.

11. The system of claim 1, wherein the coupler is positionable between the external charger and the implantable medical device.

12. The system of claim 1, wherein the coupler is positionable behind the implantable medical device from the vantage of the external charger.

13. The system of claim 1, wherein the coupler resonates when receiving the first AC magnetic field, and wherein such resonance generates the second AC magnetic field.

14. The system of claim 1, wherein the external charger is configured to generate the first AC magnetic field automatically upon detecting the implantable medical device.

15. The system of claim 1, wherein the third coil in the implantable medical device is further configured to receive the first AC magnetic field to charge the battery.

16. The system of claim 1, wherein the coupler further comprises a capacitor coupled to the second coil.

17. A system for charging a battery in an implantable medical device of a patient, comprising:
an external charger configured to emit a first AC magnetic field; and
a circuit configured to receive the first AC magnetic field and to resonate to generate a second magnetic field in response for charging the battery in the implantable medical device.

18. The system of claim 17, wherein the circuit is not powered from a power source.

19. The system of claim 17, wherein the circuit comprises a coil coupled in parallel to a capacitor.

20. The system of claim 17, wherein the first AC magnetic field and the second AC magnetic field have the same frequency.

21. The system of claim 17, wherein the external charger comprises automatic tuning circuitry for tuning the frequency of the first AC magnetic field.

22. The system of claim 17, wherein the external charger is hand-holdable and portable.

23. The system of claim 17, wherein the external charger is stationary.

24. The system of claim 17, wherein the circuit is portable.

25. The system of claim 17, wherein the circuit is integrated into a home furnishing.

26. The system of claim 17, wherein the external charger is integrated into a home furnishing.

27. The system of claim 17, wherein the circuit is incorporated into an article wearable by the patient.

28. The system of claim 17, wherein the circuit is positionable between the external charger and the implantable medical device.

29. The system of claim 17, wherein the circuit is positionable behind the implantable medical device from the vantage of the external charger.

30. The system of claim 17, wherein the external charger is configured to generate the first AC magnetic field automatically upon detecting the implantable medical device.

31. The system of claim 17, wherein the first AC magnetic field is further for charging the battery in the implantable medical device.

32. A device for facilitating charging of a battery in an implantable medical device of a patient, comprising:
an article positionable in proximity to the patient's implantable medical device, the article comprising a coupler comprising a coil, wherein the coil is configured to receive an first AC magnetic field from an external charger to generate a second AC magnetic field in response for charging the battery of the implantable medical device.

33. The device of claim 32, wherein the first AC magnetic field and the second AC magnetic field have the same frequency.

34. The device of claim 32, wherein the article is wearable by the patient.

35. The device of claim 34, wherein the wearable article is configured to hold the external charger.

36. The device of claim 35, wherein the wearable article is further configured to hold the external charger proximate to the coil of the coupler.

37. The device of claim 36, wherein the wearable article holds the external charger within an area bounded by the coil of the coupler.

38. The device of claim 32, wherein the article further comprises a pocket configured to hold the external charging device.

39. The device of claim 38, wherein the pocket is configured such that the coupler is positioned between the external charger and the implantable medical device.

40. The device of claim 32, wherein the coupler is not powered from a power source.

41. The device of claim 32, wherein the coupler resonates when receiving the first AC magnetic field, and wherein such resonance generates the second AC magnetic field.

42. The device of claim 32, wherein the article comprises a belt wearable around the patient's waist.

43. The device of claim 32, wherein the article comprises a shoulder harness for facilitating the charging of a battery in a chest-positioned implantable medical device.

44. The device of claim 32, wherein the coupler further comprises a capacitor coupled to the coil.
